(19) United States
(12) Patent Application Publication
(10) Pub. No.: US 2003/0216635 A1
(43) Pub. Date: Nov. 20, 2003

(54) Method and system for monitoring pancreatic pathologies

(76) Inventors: Irun R Cohen, Rehovot (IL); Hadassa Degani, Rehovot (IL); Lior Bruker, Modi'In (IL); Raanan Margalit, Yohanan (IL)

Correspondence Address: WINSTON & STRAWN, PATENT DEPARTMENT, 1400 L STREET, N.W., WASHINGTON, DC, 20005-3502, US

(21) Appl. No.: 10/169,466

(22) PCT Filed: Jan. 7, 2001
(86) PCT No.: PCT/IL01/00015

(30) Foreign Application Priority Data
Jan. 6, 2000 (US) 60174663

(51) Int. Cl. A61B 5/05

(52) U.S. Cl. 600/410

(57) ABSTRACT

A method for non invasively detecting and monitoring pancreatic pathologies preferably related to vascular changes or inflammatory processes in the pancreas, such as the onset of IDDM, by magnetic resonance imaging (MRI) is disclosed. The method enables the detection of IDDM prior to the appearance of clinical manifestation, by detecting early stages of IDDM such as insulitis. The disclosed method also enables correlation of different stages of pancreatic diseases with the characteristics of contrast enhancement curves. A MRI system for monitoring pancreatic pathology in a patient is also disclosed. the system comprises a single volume coil for transmitting and receiving signals from an internal body organ of a patient, such as the pancreas, or the spleen.

### FIELD OF THE INVENTION

The present invention relates to the general field of Magnetic Resonance Imaging (MRI) of body tissues. More specifically, the present invention relates to a method and system for magnetic resonance imaging of body organs and for monitoring, by MRI, pancreatic pathologies.

### BACKGROUND OF THE INVENTION

Magnetic Resonance Imaging (MRI) is a method for producing images based on spatial variations in the phase and frequency of the radio frequency (RF) energy being absorbed and emitted by an imaged object. MRI is, in fact, a special form of multidimensional Nuclear Magnetic Resonance (NMR) spectroscopy. The difference between the two is that multidimensional NMR spectroscopy resolves the inherently different resonance frequencies that characterize the different spin populations in the sample, whereas in a typical MRI procedure we are dealing, initially, with a uniform population (i.e. a single resonance frequency) that is converted deliberately to a spin ensemble with spatially dependent frequencies. The procedure creates a map of intensities vs. frequencies that is easily translated to a real image (a map of intensities vs. spatial location). The MRI procedure creates an environment that associates a spatially dependent resonance frequency to every point in space. This is done by the application of magnetic field gradients with a known dependence between the field strength and the location (hence a known functional relation between resonance frequency and location).

The MR image is a two dimensional matrix in which each point in a defined Z plane—called a voxel—has 2 coordinates (x,y) and a value that represents its intensity. This intensity is determined by the intrinsic parameters of the sample (relaxation times) and by the parameters of the procedure.

The MRI procedure includes three magnetic field gradients of the type:

B(r)=B(0)+Gᵣr (1.1)

where:

Gᵣ—gradient strength (gauss/cm)

r—any one of the three spatial axes—usually the principal axes (cm).

The application of the gradient in the Z direction along with a modulation in the envelope of the RF pulse (whose basic frequency is the Larmor frequency of the imaged spin population) leads to the selection of a specific slice in this direction. This pulse affects only those nuclei that fall in the frequency range of the modulations' Fourier Transform (FT) (centered at the Larmor frequency). But in the presence of a gradient this frequency band is, at the same time, a spatial slice along the Z direction.

As for the X and Y directions, once the slice is selected, a 2D NMR procedure is carried out in the X-Y plane, with one directional gradient turned on during the evolution time (in a “phase encode” manner) and the second during the acquisition time. The time domain data is stored in a 2D matrix, which is converted by a 2DFT to an image. This process can be summarized as follows:

Slice selection (Z)+2D experiment (XY) [symbol] 2D time domain data

[symbol]

[symbol]

Intensity=intensity (wₓ, w_{y}) [symbol] intensity=intensity(x,y,z)=image

The general form of the voxel intensity is given in the following equation:

$$Λ Λ I = A ρ sin θ ( \frac{1 - exp ( - TR / {T}_{1} )}{1 - cos θexp ( - TR / {T}_{1} )} ) exp ( - TE / {T}_{2} ) \qquad ( 1.2 )$$

where:

A—Proportion constant.

θ—The nominal flip angle of the RF pulse (degrees).

ρThe spin density in the voxel.

TR—The time between successive measurements in the 2D time domain matrix (sec).

TE—The duration of a single measurement (sec).

T₁—Longitudinal relaxation time (sec).

T₂—Transverse relaxation time (sec).

The human body is primarily fat and water. Fat and water have many hydrogen atoms which make the human body approximately 63% hydrogen atoms. Hydrogen nuclei have an NMR signal. For these reasons magnetic resonance imaging primarily images the NMR signal from the hydrogen nuclei. Each voxel of an image of the human body contains one or more tissues.

Body tissues are some times imaged using contrast enhanced MRI. This procedure involves the use of contrast agents, which are paramagnetic ions that have the ability to change the relaxation times of magnetic nuclei that interact with them.

The pancreas, one of the largest secretory glands in the human body, is situated in the upper part of the abdomen (in a cavity that lies between the spleen, the stomach and the colon) and constitutes about 0.1% of adult body mass. The pancreas can be divided functionally into two different sub-organs: the exocrine pancreas and the endocrine pancreas. The former constitutes the major mass of the gland (>95%). Its physiological role is to secrete digestive enzymes into the alimentary tract, thus helping to digest nutrients. The endocrine pancreas is composed of a large number of small cell clusters—called “The islets of Langerhans”—that are embedded in the mass of the exocrine pancreas. These islets make up only 1-2% of the gland volume. The islets are not distributed uniformly throughout the pancreas. The islets of Langerhans contain four distinct types of cells, each secreting a different hormone. The orchestrated secretion of this ensemble of hormone is aimed at controlling the exploitation of nutrients, particularly glucose. The most important hormone in this respect is insulin which is secreted from the beta cells, which account for about 75% of the islet mass. The islets are highly vascularised and account for approximately 10% of the pancreatic blood flow (Homo-Delarch, F., Boitard, C. (1996) Immunology today, 17, 456-460).

A well known and wide spread pancreatic pathology is IDDM (Insulin Dependent Diabetes Mellitus), also known as type 1 diabetes (and formerly as juvenile onset diabetes), which is a metabolic disorder that results from an insufficient (or in many cases, a complete lack of) insulin production.

By nature, the disease is autoimmune and is caused by the destruction by the immune system of the insulin producing beta cells, which are located in the islets of Langerhans in the pancreas. An untreated diabetic patient can reach the state of acute hyperglycemia and eventually coma and death (unless treated immediately with insulin). Yet, even the balanced IDDM patient who receives regular insulin injections is prone to chronic complications that stem, probably, from changes in the patient's blood vessels. One of the major cellular events in the progression of IDDM is the invasion of immune cells into the islets of Langerhans, which causes the inflammatory process called insulitis (Bach, J. F. (1994) Endocrine reviews. 15, 516-535).

There are indications that various changes in the microvasculature of the islets take place prior to the appearance of insulitis (Papaccio, G. (1993) Histol histopath. 8, 751-759).

A possible treatment for IDDM has emerged recently (Elias, D., Cohen, I. R., (1994) THE LANCET. 343, 704-706). The effectiveness of this treatment is not limited to the pre-clinical situation (in which the treatment takes a form of “vaccination”) but also to the early stages of the disease itself (the first signs of hyperglycemia). IDDM in humans does not follow a preset timetable and there is no efficient way that enables assessment in advance of which individuals will develop the disease. The existence of the disease in a human patient is diagnosed only after the appearance of clinical symptoms, at which stage most of the insulin producing cells have already been destroyed. Applying the treatment at this stage, will, at most, “rescue” 10-20% of the islets, and leave the patient with only a marginal insulin production capability.

The current situation, that combines the existence of a novel therapy, and the urgent need to give it to a patient as soon as possible, calls for a new monitoring method that will enable the detection of IDDM at its very beginning.

Attempts made up till now for the measurement of inflammatory processes in the pancreas were either limited to the much larger exocrine pancreas (Outwater, E. C., Mitchell, D. G. (1996) Topics in magnetic resonance imaging. 8, 248-264), or used invasive measures such as imaging of islet insulitis with radiolabelled immunoglobulines or cytokines (Barone, R., Procaccini, E., Chianelli, M., Anovazzi, A., Fiore, V., Hawa, M., Nardi, G., Ronga, G., Pozzilli, P., Signore, A. (1998) Eur. Jur. Nucl. Med. 25, 503-508 and Signore, A., Picarelli, A., Chianelli, M., Biancone, L., Anovazzi, A., Tiberti, C., Anastasi, E., Multary, G., Negri, M., Pallone, F., Pozzilli, P. (1996) J. pediatr. Endocrinol. Metab. 9, 139-144).

The pancreas is considered to be one of the most difficult organs to image in humans due to its location and diffuse nature. To date there exists no diagnostic method for non invasively monitoring inflammatory processes, such as the onset of IDDM or other pathologies in the pancreas.

### SUMMARY OF THE INVENTION

The present invention provides a novel system and method for non invasively detecting, as well as diagnosing and monitoring pancreatic pathologies in a patient, preferably pathologies related to vascular changes or inflammatory processes in the pancreas, such as the onset of IDDM. The present invention enables the detection of IDDM prior to the appearance of clinical manifestation, by detecting early stages of IDDM (such as insulitis). The method of the invention enables correlation of different stages of pancreatic diseases with the characteristics of contrast enhancement curves.

Thus, the present invention provides, in accordance with an embodiment of the invention, a method for monitoring a pancreatic pathology. In another embodiment the method is for detecting the occurrence of insulitis. The method according to an embodiment of the invention comprises the steps of: 1. obtaining a first magnetic resonance image of an internal body organ, such as the pancreas or the spleen, using defined sequence parameters; 2. injecting a contrast agent to the subject; 3. obtaining a plurality of subsequent contrast enhancement images of the internal body organ using the defined sequence parameters; 4. creating an intensity curve, by plotting intensity over time, from the plurality of subsequent contrast enhancement images; 5. converting the intensity curve to an enhancement curve, the enhancement curve having a linear portion and a plateau portion; 6. extracting an enhancement value at plateau from the enhancement curve; and 7. comparing the enhancement value at plateau to a standard. The comparison provides information regarding the pathology, thereby making it possible to monitor the pancreatic pathology in the subject. In this embodiment it is preferable to obtain a large portion of the subsequent contrast enhancement images at a time correlating to the plateau portion of the enhancement curve.

In another embodiment of the invention steps 6 and 7 may be replaced with the steps of extracting an initial rate value of the enhancement curve; and comparing the initial rate value to a standard. In this embodiment it is preferable to obtain a large portion of the subsequent contrast enhancement images at a time correlating to the linear portion of the enhancement curve.

Optionally, for purposes of localizing the internal body organ, an axial image of the internal body organ can be obtained prior to the step of obtaining a first magnetic resonance image. The axial image has defined alignment parameters and the step of obtaining a first magnetic resonance image and the step of obtaining a plurality of subsequent contrast enhancement images are preformed by using the same defined alignment parameters. Obtaining the axial image may be done by applying to the internal body organ a fat suppression pulse having a determined pulse offset frequency and a determined bandwidth and then obtaining a T1 gradient echo image of the internal body organ.

Preferably, the contrast agent is unable to intersect cell membranes and can not enter cells and is thus restricted to the extracellular space. The contrast agent may be, for example, gadolinium diethylenetriamine pentaacetic acid. Preferably, the contrast agent is injected intravenously (IV) to the subject.

The present invention further provides an MRI system for monitoring a pancreatic pathology in a subject. The system comprises a single volume coil for transmitting and receiving signals from an internal body organ, such as the pancreas or the spleen. The system may also comprise a spectrometer recording at 4.7 Tesla.

### BRIEF DESCRIPTION OF THE FIGURES

The present invention will be understood and appreciated more fully from the following detailed description taken in conjunction with the appended drawings in which:

FIG. 1 is a graphic presentation of a s/n comparison between two software versions in accordance with an embodiment of the invention;

FIG. 2 is a T₁ weighted gradient echo axial image recorded with a volume coil;

FIG. 3 is a graphic presentation of the s/n values in an examined frequency range;

FIG. 4 is a graphic presentation of contrast values in an examined frequency range;

FIGS. 5A and 5B present T₁ weighted gradient echo images of a NOD female mouse: A. without fat suppression, B. with fat suppression;

FIG. 6 is a graphic presentation of the simulated enhancement curves for eight different TR values using a flip angle of 30 degrees;

FIG. 7 is a graphic presentation of the simulated enhancement curves for nine different flip angles using a TR value of 20 msec;

FIG. 8 is a graphic presentation of the simulated enhancement curves for nine different flip angles using a TR value of 150 msec;

FIG. 9 is a graphical presentation of the comparison of enhancement vs. [Gd] curves for 7 different TR times;

FIG. 10 is a graphical presentation of the comparison of enhancement vs. [Gd] curves for two extreme TR values using two different flip angles in each case;

FIG. 11 shows plot of maximal spleen enhancement vs. blood glucose levels in 4 BALB/c mice;

FIG. 12 shows a plot of maximal spleen enhancement vs. blood glucose levels for 10 NOD mice; and

FIG. 13 is a histogram presentation of the mean of the maximal spleen enhancement classified into three animal groups.

FIG. 14 is a histogram presentation of the association of the mean “a value” with the histological condition of the pancreas;

### DETAILED DESCRIPTION OF THE INVENTION

The present invention will be further described and demonstrated by the following experimental procedures. It should be appreciated that the examples and experiments described herein are not intended to limit the scope of the invention but rather to illustrate and exemplify the method and system of the invention.

Experiments aimed at harnessing MRI to the monitoring of IDDM development are described.

The NOD Mouse—An Experimental Model for Human IDDM

The present invention was triggered, inter alia, by the discovery of a new therapy for IDDM, as described above. The efficiency of this therapy in NOD (Non Obese Diabetic) mice was proven to be very high, provided it is given very early in the course of progression of the disease—well before its clinical manifestations. This constraint created a need for a new diagnostic method for IDDM that could monitor the disease progression, and provide an early detection, as well as diagnosis and monitoring of treatment. The current knowledge of the IDDM process, suggested that a suitable candidate for monitoring—i.e. a mechanism that undergoes a detectable change from the early stages of the disease—is the marked inflammatory change that take place in the pancreas (mainly in its endocrine part).

The most popular animal model for the investigation of human IDDM is that of the NOD (Non Obese Diabetic) mouse. Developed in the late '70 (initially for a different purpose), this strain of mice showed a spontaneous type of diabetes that is very similar to the human IDDM. As in humans, the NOD IDDM is a multifactorial autoimmune disease that is under the control of many (>15) genes. It also shares the same histological-functional course as human IDDM, going from periinsulitis to insulitis, selective destruction of beta cells and finally to the clinical picture of blood hyperglycemia. The only marked differences between human and NOD IDDM are the female predominance and the low level (compared to humans) of islet-reactive autoantibodies in the NOD mice. The development of the disease in the NOD strain follows a specific timetable, as follows: the onset of insulitis (at the age of 4 weeks), followed by hyperglycemia (14-17 weeks of age) and finally severe diabetes (weeks 35-40). The existence of such a known timetable of events makes this strain even more suited for research.

MRI—Experimental Setup

The MRI experimental setup includes three magnetic field gradients as discussed above. The existence of the applied magnetic field gradients causes a dephasing of the detected signal. Hence, it is not customary to detect the time domain signal as a simple FID (Free Induced Decay), but rather as an echo that is created in such a manner as to rephase the signal. In principal there are two main methods of creating an echo:

1. The “GRADIENT ECHO” method, in which additional gradients with opposite signs are turned on during the experiment, which will rephase the signal at the time of acquisition TE.

2. The “SPIN ECHO” method, in which, in addition to the gradient rephasing, there is also a rephasing of the background inhomogeneities (B₀ inhomogeneities). This is done by setting the first RF (Radio Frequency) pulse to be a 90° pulse and adding a 180° pulse at TE/2. As a result at the acquisition time TE, both rephasing mechanisms will coalesce to create the true signal.

In most cases the Spin Echo technique creates more intense signals and therefor images with superior s/n ratios compared to Gradient Echo images. On the other hand, in the Gradient Echo sequence, one can use flip angles smaller than 90°. This results in much shorter TR values (and therefore also shorter imaging times). The sample's intrinsic parameters can be used to create three “classes” of images by weighting most of the signal intensity according to only one of the parameters each time. More elaborately:

1. “T1 weighted” images are obtained by shortening TE to a minimum and choosing the TR to be of the order of T₁ (but smaller, to gain a better s/n ratio per unit time).

2. “T2 weighted” images are obtained when T₁<<TR, while TE is of the order of T₂.

3. “Density weighted” images require minimizing TE and maximizing TR compared to T₂ and T₁ respectively.

These “weighting” measures are especially useful when imaging an anatomical specimen. The image results from the inherent differences between tissues with regards to T₁ and T₂ values (due to different water content, presence of paramagnetic ions, etc.).

Contrast Enhanced MRI

Contrast agents are paramagnetic ions that have the ability to change the relaxation times of magnetic nuclei that interact with them. By doing so, they afford the opportunity to change in a selective manner the intensity of certain regions in a sample. The change in the relaxation times is proportional to the concentration of the contrast agent:

$$K K \frac{1}{{T}_{1}} = \frac{1}{{T}_{1}^{0}} + R [ {C}_{t} ] \qquad ( 1.3 )$$

* The transverse relaxation time T₂ is changing in a similar way. where:

T₁—Longitudinal relaxation time with the contrast agent (sec).

T⁰₁—Original longitudinal relaxation time (sec).

R—Relaxivity constant (mM⁻¹sec⁻¹).

[Cₜ]—Contrast agent concentration [mM].

Substitution of equation (1.3) into the above mentioned equation (1.2), yields immediately a dependence of the intensity on the contrast agent's concentration:

$$Λ Λ I = f ( [ {C}_{t} ] ) = A ρ sin θ ( \frac{1 - {ⅇ}^{- TR ( 1 / {T}_{1}^{0} + R [ {C}_{t} ] )}}{1 - cos θ {ⅇ}^{- TR ( 1 / {T}_{1}^{0} + R [ {C}_{t} ] )}} ) {ⅇ}^{- \frac{TE}{{T}_{2}}} \qquad ( 1.4 )$$

Therefore, the analysis of the intensity change in a tissue before and after the administration of a contrast agent can serve to determine the value of certain tissue parameters that govern the concentration of the contrast agent in that tissue.

One of the most widely used contrast agents in ¹H imaging is a Gadoliniun complex—termed GdDTPA (gadolinium-diethylenetriamine-pentaacetic-acid)—that interacts with the water protons and shortens their relaxation times. Physiologically, this agent can travel back and forth between the blood vessels and the extracellular space but can not enter through the cell membrane into cells. In parallel to entering the body tissues, GdDTPA is filtrated out constantly from the kidneys into the urine. As a result of these pharmacokinetics, there is also a change of intensity over time (according to equation 1.4) in the body images. Consequently, one can define and record dynamic “intensity profiles” of an image over time after an injection of a contrast agent (i.e. GdDTPA). It can be assumed that the contrast agent's concentration in a given tissue is dependent on, at least, two histological parameters. These are:

1. The average extracellular volume fraction (which is the space available for the Gd complex within the tissue boundaries).

2. The product of the blood vessels surface area by their permeability to the contrast agent in the tissue (which is a measure of the agent's ability to “leak” from the blood vessels into the tissue).

In other words:

[Cₜ]=g(time, extracellular volume, permeability·surface area, flow). (1.5)

* when permeability is rate limiting relative to the flow, the latter can be neglected. Although they are a rich source of information, intensity profiles of a tissue suffer from the disadvantage of not being normalized. In other words, intrinsic differences between different tissues (i.e. in relaxation times), or even statistical diversity in the parameters of the same tissue within an animal group, could change the pattern of the intensity profile even if the concentration over time of the contrast agent in the tissue is the same. In order to overcome this problem, it is customary to convert the intensity profile to a normalized form of enhancement which is defined as:

$$Λ Λ E = \frac{I - {I}_{0}}{{I}_{0}} \qquad ( 1.6 )$$

Where I₀ and I are the tissue's intensities pre and post injection of a contrast agent, respectively. Clearly, the enhancement function is also sensitive to the tissue parameters that appear in equation (1.5).

Fat Suppression Techniques

In many biological samples, in particular in the case of ¹H imaging, there are two widespread spin populations: the water protons (in most cases the desired population), and the fat protons. The latter is close in frequency (3.5 ppm) to that of the water protons, hence the RF pulse, which is rather broadband, excites also the fat protons. This could be a disadvantage in cases where it is not desirable for the fat to appear in the image. Moreover, the computerized algorithm interprets the fatty regions, which have an inherently different resonance frequency, as if their frequency arises from their location (due to the magnetic field gradients), resulting in an image artifact (a false location of the fatty regions in the image).

One of the major classes of techniques that were devised to eliminate the fat from the final image is based on the difference in the resonance frequencies between the water and the fat protons. The key element in this group of “fat suppression” methods is the use of a selective narrow band pulse—centered on the fat frequency—prior to the regular RF pulse. The former interacts with the fat protons in one of several ways (excitation or saturation) such that the regular RF image will excite only the water protons (thus the final image will be attributed only to the water protons).

The specific method that was used in the present invention is that of “selective excitation”. In this method, a narrow 90° selective pulse rotates the fat magnetization to the x-y plane. The immediate application of a magnetic field gradient (a “spoiling gradient”) disperses the ensemble of fat magnetization in the x-y plane and results in a zero net magnetization. Meanwhile the unexcited water magnetization stays in the z direction and is subsequently imaged in one of the regular imaging sequences.

The Experimental Setup

An imaging sequence of the T1 weighted Gradient echo type was carried out for imaging NOD mice pancreas. The mouse pancreas was assumed to have a T1 of about 1 second in the set up of the invention (at 4.7 Tesla). This was extrapolated from a pancreatic T1 in humans of about 500 milliseconds at 1.5 Tesla (Outwater, E. C., Mitchell, D. G. (1996) Topics in magnetic resonance imaging. 8, 248-264).

Materials and Protocols

1. Hardware—All images were recorded at 4.7 Tesla using a Bruker Biospec 4.7/30 spectrometer. The RF coil was a Bruker volume coil with a diameter of 7.5 cm. The surface coil, when used, was a Bruker coil with a diameter of 2.5 cm. Gradient hardware consisted of unshielded gradient coils with a maximum gradient strength of 48.4 mTesla/meter with a rise time of 500 msec, using a standard gradient pre-emphasis installed by the manufacturer.

2. Software—Spectrometer operation and image analysis were done with version 2.0 of the Bruker ParaVision software, unless otherwise specified.

3. In vitro (“Phantom”) model—A phantom model was used for optimization. This was composed of small vials taped together, containing solutions of GdDTPA (Schering, Berlin, Germany) in saline in the range of 0-1.66 mM.

4. Animal model—In vivo images were done on female NOD mice taken from the NOD colony of Prof. Irun Cohen (Department of Immunology, the Weizmann Institute, Rehovot, Israel).

5. Anesthesia—In this section, animals were anesthetized with a mixture of 85% Ketaset (Ketamine) and 15% Xylazine (taken from a stock solution of 2%). Out of this mixture 40 μl were injected I.P. Later on it turned out that this anesthetic mixture has a dramatic influence on the blood Glucose levels. Consequently all enhancement measurements were done using another anesthetic (see below). The identity of the anesthetic was of no importance during the optimization experiments.

Results

Creating a Multi-concentration Phantom

The proposed contrast enhanced measurements are carried out under a varying Gd concentration—a maximal concentration right after the injection that decays gradually to zero due to the agent's clearance through the kidneys. Consequently, when aiming to optimize the working parameters in the above outlined manner, a model had to be devised that could simulate the behavior of the pancreas within this concentration range. Moreover, since the weighting method was about to be of the T₁ type, this model should also reflect the true value of T₁ in the pancreas in every concentration. Looking at equation (1.3) and substituting the following values

T₁⁰_{pancreas}=1 sec; T₁⁰_{water}=3.5 sec; R_{Gd}=4.3 mM⁻¹ sec ⁻¹

We obtain:

$$\begin{matrix}Λ Λ \frac{1}{{T}_{1 pancreas}} = 1 + {4.3 [ Gd ]}_{pancreas} & ( 3.1 ) \\ Λ Λ \frac{1}{{T}_{1 water}} = \frac{1}{3.5} + {4.3 [ Gd ]}_{water} & ( 3.2 )\end{matrix}$$

When T_{1water}=T_{1pancreas}, then equating both equations yields:

Λ[Gd]_{pancreas}+0.166=[Gd]_{water} (3.3)

Hence, in order to simulate the T₁ value of the pancreas in a water solution a Gd concentration in the phantom that is higher by 0.166 mM than that in the pancreas, should be used.

As For the actual concentration of the contrast agent in the pancreas, one can take as an upper limit (which is considerably higher than the true upper concentration) the concentration of the contrast agent in the blood immediately after an I.V. injection.

In the present invention, the estimated bolus injection was of 200 μliter taken from a 0.05M Gd solution. Assuming that the total blood volume of a mouse is about 5 ml, we get:

[Gd]ₘₐₓ=(200*10⁻⁶*0.05)/(5*10⁻³)=0.002 M=2 mM

Hence, for all practical purposes it can be assumed that the Gd concentration in the pancreas ranges from 0 to 1.5 mM.

Experimenting with the Basic Elements of the Setup

In the preliminary part of the research, the basic elements of the setup were chosen in a way that would maximize the s/n ratio and facilitate the localization of the pancreas. More specifically, two versions of the Bruker ParaVision software and two different receiving coils (a volume coil vs. a surface coil) were compared.

Software Comparison

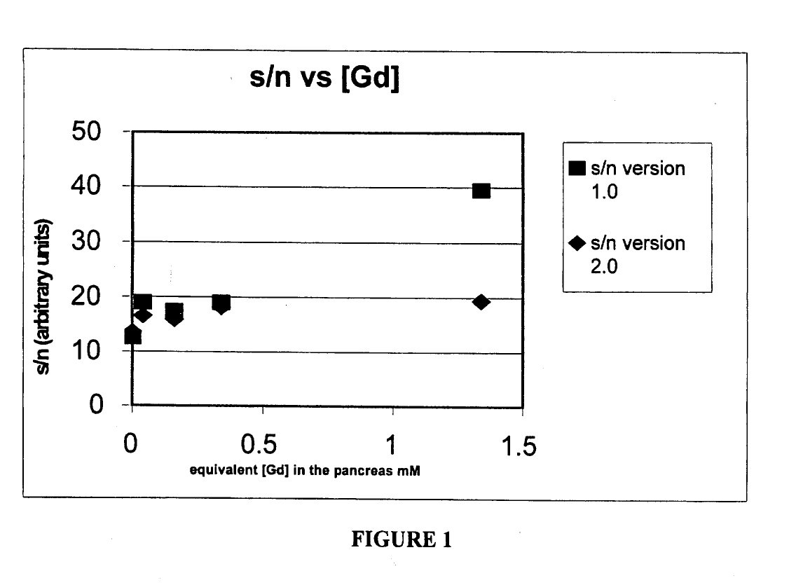

A new version (version 2.0) of the ParaVision software was introduced by Bruker at the time of the experiments. In order to compare the s/n ratio between the versions, the multi-concentration phantom was used with concentrations of 0.16, 0.2, 0.3, 0.5, 1.5 mM in saline that corresponded (according to equation 3.3) to equivalent pancreatic concentrations of 0, 0.04, 0.14, 0.34, 1.34 mM, respectively. Signal intensities were averaged over the cross-section of each vial. The noise level was taken as the standard deviation of a comparable region outside the phantom. The results are summarized in FIG. 1, which is a graphic presentation of an s/n comparison between two software versions. Images were recorded with Bruker's “GEFI” sequence, TR/TE=80/5 msec, fov=4×4 cm, matrix size=256×256, and number of averages=2. Ignoring the extreme point of 1.34 mM (that suffered from a folding effect), both software versions showed comparable s/n ratios. As a result, version 2.0, which was superior in other aspects, was chosen to work with.

Coil Configuration Comparison

The next step was to compare two different coil configurations. In the first configuration, a single volume coil served as both a transmitter and a receiver. In the second configuration, a volume coil served as the transmitter, but the signal was received by a surface coil attached to the sample. The latter configuration had the advantage of an improved s/n ratio in the vicinity of the coil. Yet this s/n is inversely proportional to the distance from the coil and decays rapidly with distance. In addition, the imaged slices in the surface coil configuration are limited to slices with a parallel orientation with respect to the coil's plane. A female NOD mouse served as a sample in both configurations. The aim was to compare the images in two aspects:

1. The “slice quality” (i.e. how easy is it to localize the pancreas, how many pancreatic pixels are present in the image).

2. The s/n ratio.

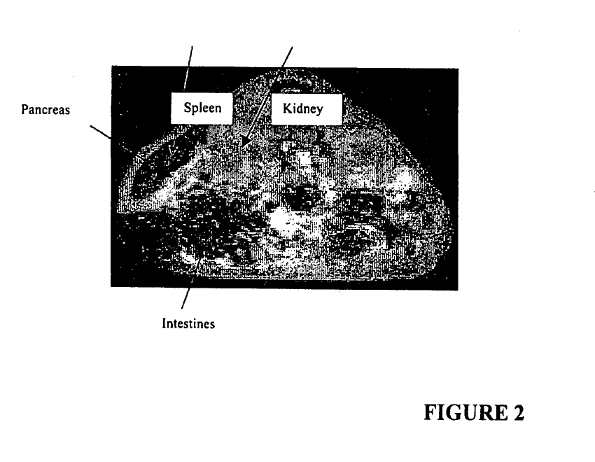

Two typical images recorded in both configurations are shown in FIG. 2.

FIG. 2 is a T₁ weighted gradient echo axial image recorded with a volume coil. The image was recorded with a “GEFI” sequence TR/TE=150/5 msec, flip angle=30 deg, matrix size=256×256, fov=4×4 cm, number of averages =8. One can readily observe that the first image (FIG. 2) is superior with respect to the “slice quality” parameter. It contains a larger portion of the pancreas and several “anatomical markers” (the spleen, kidney and intestines) that surround the pancreas in an orderly fashion. Moreover, this configuration is more suited to the localization of the tail of the pancreas which is richer (at least in humans) in Langerhans Islets. In contrast, in the second configuration one is limited to coronal sections (because the coil is situated below the animal's belly), which are less suited for localization. Thus, it was decided (even without comparing the s/n ratio) to carry on with the volume coil configuration.

Improving the Ability to Localize the Pancreas

To achieve improved ability to localize the pancreas two experiments were conducted:

1. A one-time experiment in which a glass capillary, filled with water, was implanted near the pancreas of a living animal. This animal was imaged later, with the glass capillary serving as a marker.

2. The optimization and incorporation of a fat suppression pulse as a routine measure. This reduced markedly the fat signal in the image and helped in distinguishing the pancreas from its surroundings.

The Capillary Implantation Experiment

In this experiment, a thin glass capillary, filled with water, was implanted adjacent to (and above) the pancreas of a female NOD mouse. A T₁ weighted Gradient echo coronal image recorded with a surface coil was obtained (not shown). The image was recorded with a “GEFI” sequence TR/TE=160/5 msec, flip angle=30 deg, matrix size=256×256, fov=4×4 cm, number of averages=8. Under the conditions specified above, the capillary appeared as a dark line on the bright background of the surrounding fat and tissues. In order to verify the observations, this animal was later on dissected and the capillary's position was recorded. This experiment was not intended to demonstrate a high resolution “localization ability” of the pancreas—which is impossible with this crude setup. Rather, it was intended to test the ability to localize the “gross location” of the gland.

Incorporation of a Fat Suppression Pulse

The incorporation of a “fat suppression” pulse as an integral part of our working protocol was considered as an important contribution to the localization ability. More specifically a standard fat-suppression sequence (Bruker's “gefi_fat_supp_mod_bio”) was chosen whose parameters were optimized to suit the specific needs of the system and method. As discussed above, the fat suppression pulse is a 90° RF pulse (given prior to the regular pulse), which is characterized by two parameters:

1. The pulse frequency (defined practically as an offset frequency with respect to that of the water protons).

2. The pulse bandwidth.

The first parameter can be easily computed, since the desired offset frequency should equal exactly the difference in resonance frequencies between fat and water protons. When this condition is fulfilled, the fat suppression pulse is centered exactly on the resonance frequency of the fat. On the other hand, the determination of the second parameter is less trivial and can be done only by experimentation. Note that neither the fat nor the water has an ideal resonance peak “situated” on a single frequency. As a result, the fat suppression pulse should be of a considerable bandwidth in order to suppress most of the fat protons. Yet, it shouldn't be too broad, otherwise it will overlap (at least partially) with the water resonance peak and will suppress also the desired water signal. All and all, this bandwidth represents a compromise between a maximal fat suppression and minimal water suppression.

Determining the Pulse Offset Frequency

For any two given proton species, a and b, one can write:

ΛΛΔHz_{ab}=vₐ−v_{b}=v₀(ppmₐ−ppm_{b}) (3.4)

where:

ΔHz_{ab}=offset frequency (Hz)

v₀=basic resonance frequency of the protons in the spectrometer (i.e. the given B₀)

ppmₓ—the chemical shift of species x (ppm)

The spectrometer used in the experiments had v₀ of 200 MHz, and the chemical shifts of water and fat are known (4.7 and 1.2 ppm respectively), thus the following is obtained:

ΔHz_{water-fat}=700 Hz

This frequency difference was inserted as the offset frequency of the fat suppression pulse.

Determining the Pulse Frequency Bandwidth

The optimization of the bandwidth of the fat suppression pulse was carried out in four different frequencies spanning over a wide frequency range (from 500 Hz, below the frequency difference of 700 Hz, and up to 1400 Hz—way above it). For the purpose of eliminating the fat signal on the one hand, while minimizing the reduction in the water signal on the other hand, two parameters were measured:

1. The s/n ratio—defined as the signal intensity of the pancreas divided by the noise. This parameter is sensitive to the water signal.

2. The contrast—defined as the signal intensity in the pancreas divided by that of the ovary. This parameter is dependent on the fat signal and measures the ability to distinguish the pancreatic tissue from the fat tissue. The choice of the ovary stemmed from its closeness to the pancreas and the abundance of fatty tissues around it.

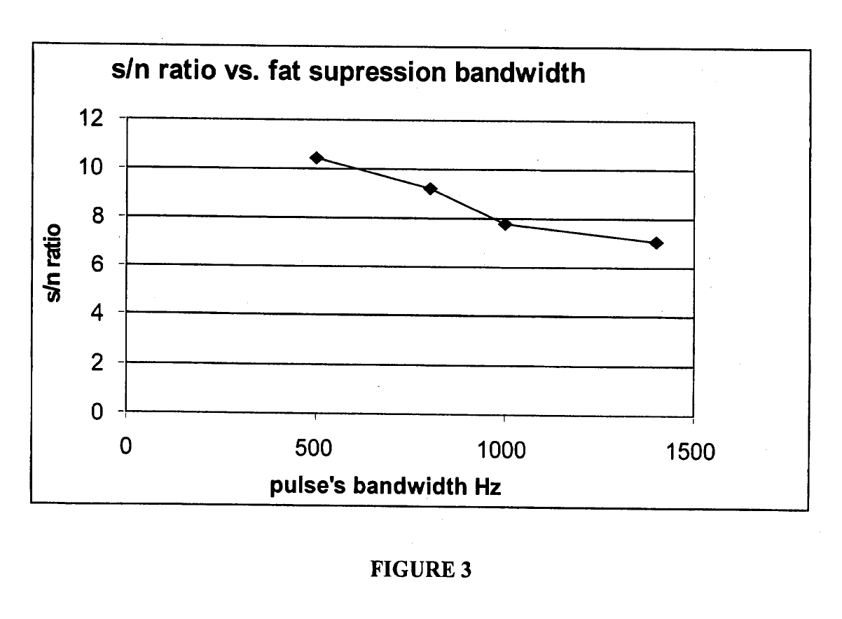

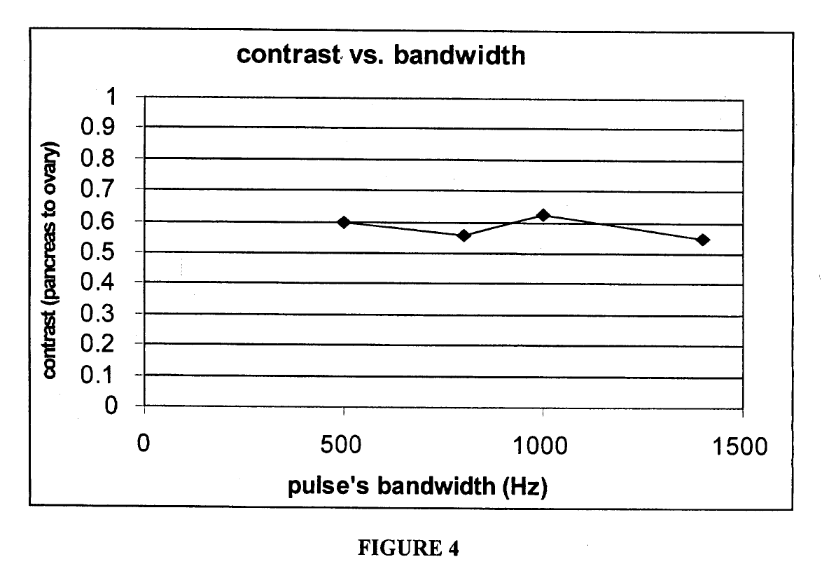

All measurements were carried out on an image of a female NOD mouse (see parameters in FIG. 3 below). The actual values of the above parameters were computed on ROI's (regions of interest) drawn in the pancreas and the ovary, using suitable computer programs. The results are summarized in FIGS. 3 and 4.

FIG. 3 shows the s/n values in the examined frequency range. A steady decrease in the s/n is shown. This decrease results from the lowering of the water signal as the fat suppression pulse grows wider and overlaps the resonance curve of the water protons.

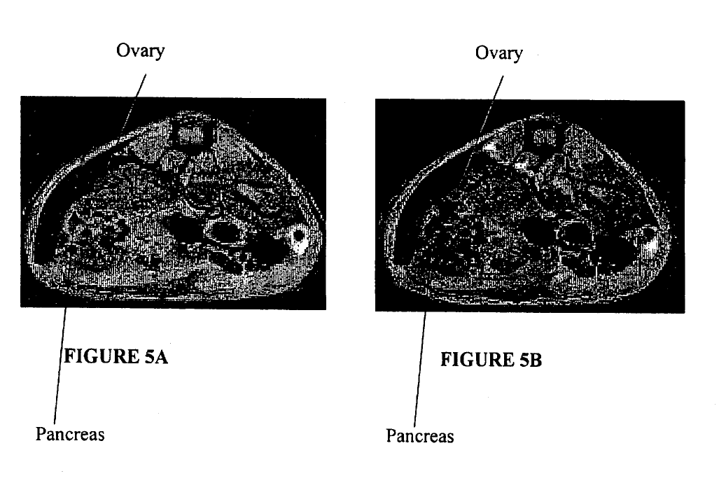

FIG. 4 shows contrast values in the examined frequency range. It can be seen that the contras values “oscillate” around a fixed value and do not show a defined trend. Examining the results shows that the s/n ratio increases as the bandwidth decreases. At the same time, the contrast value stays almost fixed over the frequency range. The conclusion was to choose a narrow bandwidth of 500 Hz for the fat suppression signal. The advantage of using a fat suppressed image is exemplified in FIGS. 5A and 5B. The axial cross section shown in FIGS. 5A and 5B is not a typical one due to the need to view considerable portions of the pancreas and ovary in the same slice. In addition, the unusual vividness of the image was achieved only because the animal died a short time before it was imaged. Nevertheless these images demonstrate the characteristics of the fat suppression method.

FIG. 5 presents two T₁ weighted Gradient echo images of a NOD female mouse. FIG. 5A is an image taked without fat suppression while FIG. 5B includes a fat suppression pulse with an offset frequency of 700 Hz and a bandwidth of 500 Hz. Other parameters are TR/TE=80/6 msec, flip angle=22.5⁰, matrix size=256×256, fov=4×4 cm. Note the elimination of the ovarian fat, which is accompanied by a general reduction in the signal intensity in the fat suppressed image (FIG. 5B).

Optimizing the Working Parameters of the Dynamic Collection

Once a satisfactory level of “pancreas localization” was reached, the parameters of the “dynamic collection”—the images taken prior to and after the administration of the contrast agent, were optimized. The optimal parameters are those in which the pancreatic enhancement curve (after the GdDTPA injection) is maximized while, at the same time, being linear over the contrast agent's concentration range. The enhancement function itself can be obtained in its explicit form by substituting equation 1.4 into equation 1.6. This yields the following expression:

$$Λ Λ E = \{ \frac{( 1 - exp ⟨ - TR / {T}_{1}^{0} ⟩ cos θ ) ( 1 - exp ⟨ - TR [ 1 / {T}_{1}^{0} + R [ Gd ] ] ⟩ )}{( 1 - exp ⟨ - TR [ 1 / {T}_{1}^{0} + [ Gd ] ] ⟩ cos θ ) ( 1 - exp ⟨ - TR / {T}_{1}^{0} ⟩ )} \} - 1 \qquad ( 3.5 )$$

(the T₂ contribution is neglected since TE/T₂=>0 for short TE). One sees immediately that the controlled parameters in this equation are TR and θ (the flip angle of the RF pulse). These are also the parameters that can be optimize to achieve the objectives of the invention. The actual optimization was done twice—once by a theoretical simulation and for the second time experimentally. In both cases, the TR values ranged from 20 milliseconds (very close to the technical limitations of the instrument—for this sequence) to 200 milliseconds (a relatively long time but still short enough to satisfy the condition of T₁ weighting, considering the T₁⁰ of the pancreas).

As for the optimization of the flip angle, the “Ernst angle”, which is defined as the flip angle yielding the highest signal in a Gradient echo image, was taken as a “marker”. This can be found by finding the derivative of equation 1.2 with respect to θ, and equating it to zero. This gives the optimal flip angle θₒₚₜ:

ΛΛθₒₚₜ=cos⁻¹(e^{-TR/T}^{₁}) (3.6)

Angles different than θₒₚₜ will give a lower signal. On the other hand, increasing the flip angle will give a higher enhancement since the magnetization “spends” more time under the T₁ weighting condition before returning to the z axis. Thus the aim is to increase the flip angle to increase the enhancement, but still keep it close to its optimal value so as not to loose in the s/n ratio. Substitution of the two extreme TR values (20, 200 msec) into equation 3.6 (assuming T₁=T₁⁰) gives optimal flip angles of 11 and 35 degrees, respectively. Thus, a basic flip angle of 30 degrees was chosen, the behavior of the system was also observed with a larger flip angle.

Theoretical Optimization

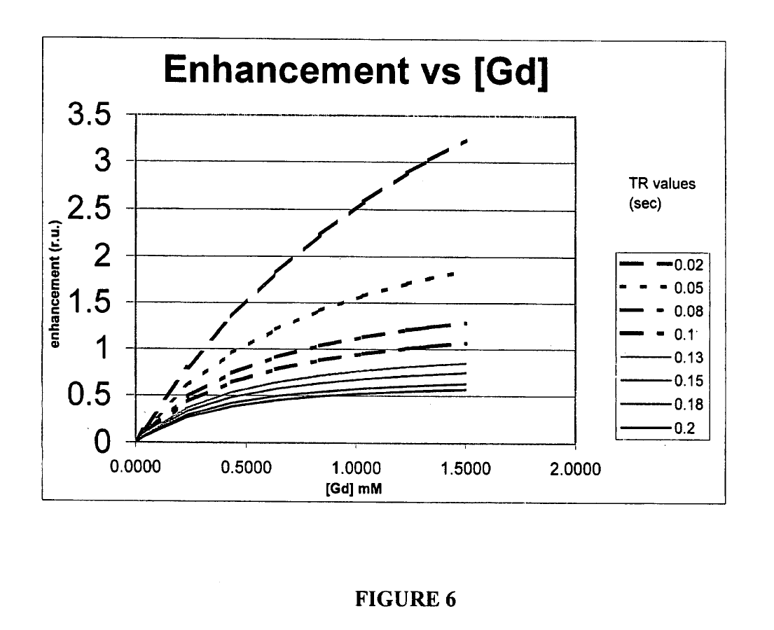

The theoretical optimization was done using MS excel software. In this simulation, the enhancement curve, according to equation 3.5, was plotted against the GdDTPA concentration up to a concentration of 1.5 mM. The enhancement curve was plotted for eight different TR values between 20 and 200 milliseconds, using a flip angle value of 30 degrees (see FIG. 6).

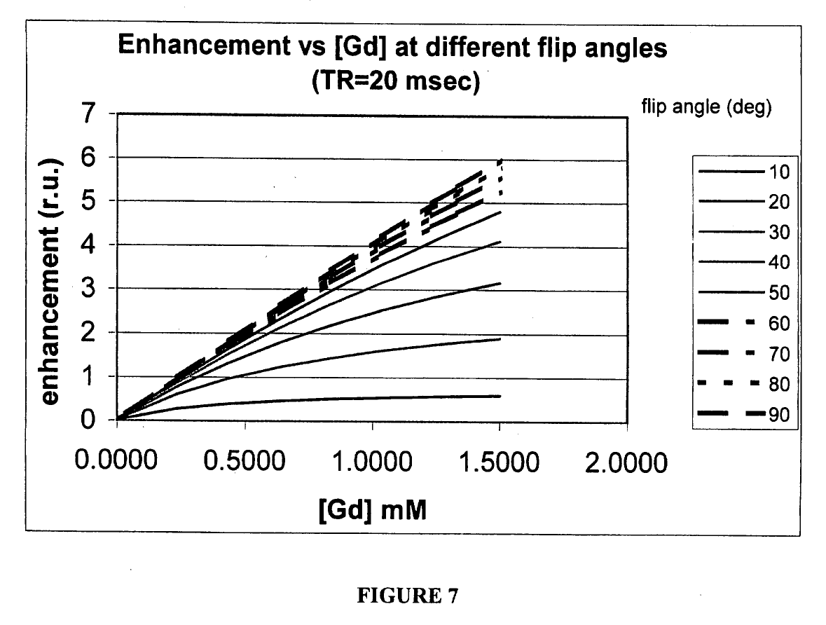

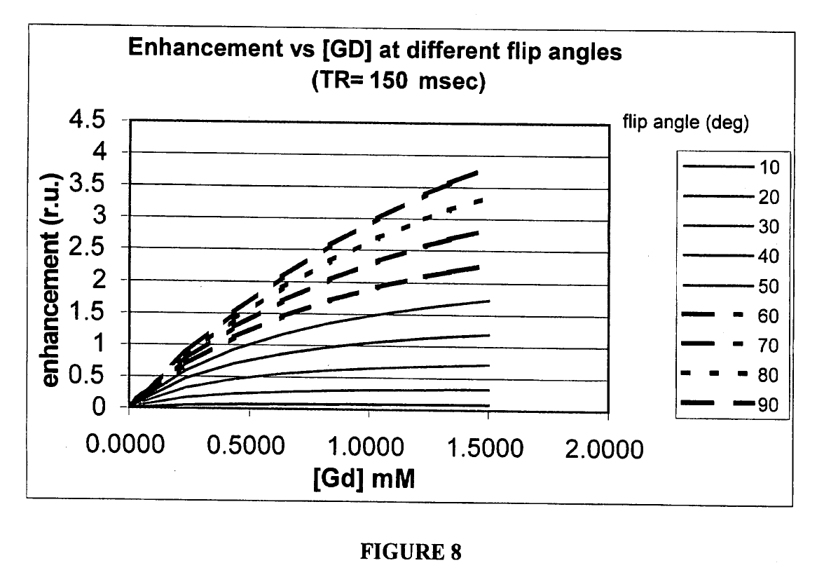

Two additional simulations demonstrated the dependence of the enhancement on the flip angle. In this case the enhancement curves were plotted for two extreme values of TR, using 9 different flip angles in the range of 10-90 degrees (FIG. 7—TR value of 20 msec, and FIG. 8—TR value of 150 msec).

These above simulations showed a clear preference toward shorter TR values, which exhibited both increased enhancement and linearity of the enhancement over most of the concentration range. As expected, larger flip angles showed the same trends.

Experimental Optimization

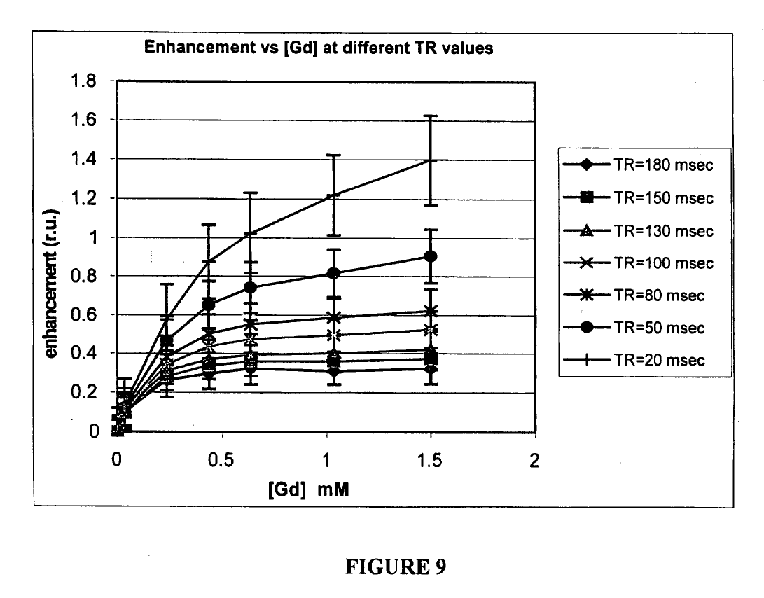

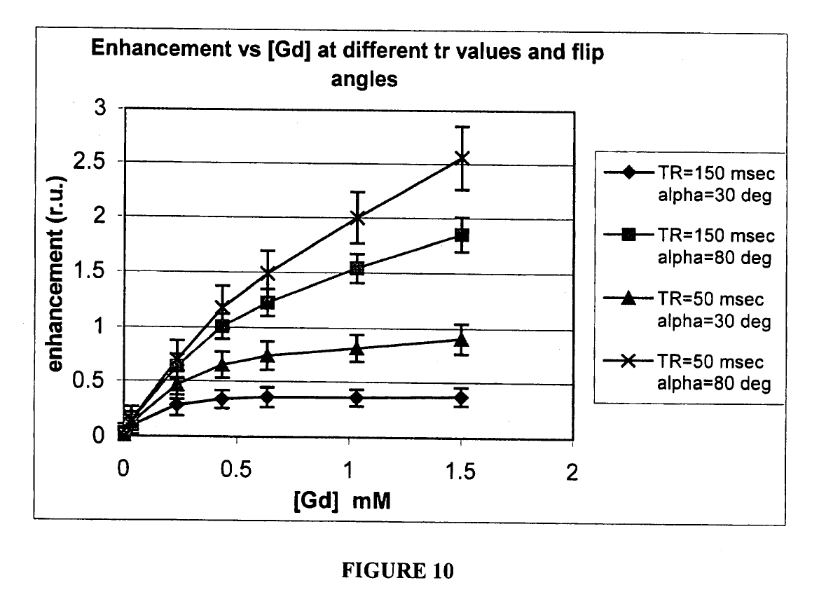

The experimental optimization was almost a repeat of the theoretical simulations with regard to the values of TR and θ. The measurements were done on a multi-concentration “phantom”, that simulated concentrations of 0, 0.03, 0.23, 0.43, 0.63, 1.03, 1.5 mM of GdDTPA in the pancreas. The sequence used was a simple Gradient echo sequence (an initial attempt to use the fat suppressed Gradient echo gave unreasonable results). The average intensity in each vial was measured using the ParaVision software. Intensity data was transferred later on to the Ms excel software and converted to enhancement values. The experimental results are summarized in FIGS. 9 and 10. FIG. 9 is a graphical presentation of the comparison of enhancement vs. [Gd] curves for 7 different TR times. The data were extracted from T₁ weighted Gradient echo images (Bruker's “gefi_bio” sequence) with the following parameters: TE=4 msec, flip angle=30 degrees, matrix size=256×256, fov=4×4 cm, number of averages=2. FIG. 10 is a graphical presentation of the comparison of enhancement vs. [Gd] curves for two extreme TR values using two different flip angles in each case. The sequence parameters are the same as in FIG. 9.

The results of the experimental optimization were in good accord with the theoretical simulation, showing an increase in the value and linearity of the enhancement with shorter TR times and/or higher flip angles. It should be mentioned though, that for some unknown reason, the enhancement values themselves were lower by a factor of —0.5 compared to the theoretical simulation.

Conclusions

The results of both optimizations pointed out clearly in favor of short TR times. Shorter TR times imply also shorter imaging times and therefore higher temporal resolution. Regarding the flip angle, a moderate flip angle, with a higher s/n ratio was preferred to a higher angle and improved enhancement. Consequently, the “dynamic collection” images were taken with TR times of 20 milliseconds and a flip angle of 30 degrees.

### CONTRAST ENHANCEMENT MEASUREMENTS AND CORRELATION WITH OTHER IDDM PARAMETERS

Experiments were carried out for the conduction of contrast enhanced imaging of the pancreas in mice, using the imaging working protocol that was consolidated on the basis of the optimization experiments described above.

Three mouse populations were examined: normal BALB/c mice (which served as a control), pre-diabetic NOD mice and diabetic NOD mice (the classification being verified by blood glucose measurements). Numerical parameters characteristic of the enhancement curve obtained for each animal were then derived from the data. The question examined is whether a clinical classification into three groups is reflected in the values of the above numerical parameters. The relation between the contrast enhancement parameters and a qualitative histological “grading” of the pancreas for each animal was also examined. In addition, the relation between the enhancement curve of the spleen in each animal and it's IDDM stage was explored.

Materials and Methods

1. Hardware—all images were recorded at 4.7 Tesla using a Bruker Biospec 4.7/30 spectrometer. The RF coil was a Bruker volume coil with a diameter of 7.5 cm. Gradient hardware consisted of unshielded gradient coils with a maximum gradient strength of 48.4 mTesla/meter with a rise time of 500 msec, using a standard gradient preemphasis installed by the manufacturer.

2. Software—intensity curves were derived from the raw images taken before and after the Gd injection, using home built computer programs (by Dov Grobgeld and Yael Paran). Intensity curves were converted to enhancement curves in MS excel. Fitting to phenomenological functions and derivation of numerical parameters was done with Microcal “origin” version 4.10 (Microcal software, USA).

3. Animal model—the mice population included either female NOD LT or female BALB/C, taken from the mice colonies of Prof. Irun Cohen (Department of Immunology, Weizmann Institute, Rehovot, Israel).

4. Glucose measurements—blood glucose measurements were done using a glucometer (Precision, Medisense) on a drop of blood taken from the animal's tail.

The measurements were conducted immediately before the imaging session of each animal.

5. Anesthesia—animals were anesthetized with a solution of Nembutal (Pental veterinary, CTS chemicals, Israel) in PBS (Dulbeco). The stock solution (60 mg/ml) was diluted 1:10, out of which 220 μl were injected I.P. to every animal. This is equivalent to a dose of 53-mg/Kg weight (assuming that a typical mouse weighs about 25 gm).

6. Histological staining—at the end of each imaging session, the pancreas of the animal was removed, fixed in a 10% formaldehyde solution and finally imbedded in paraffin. Representative 4 μm thick slices were stained with Hematoxylin-Eosin (H&E) and examined under the microscope.

The Structure of a Typical MRI Session

For the object of measuring the average enhancement of the pancreatic pixels (and those of several other organs as well) over time—before and after the injection of the contrast agent, it was required first to localize the pancreas (or other organ) and then to record a series of images of the same slice—under optimal conditions—before and after the contrast agent (i.e. Gd) injection.

Thus, the typical MRI session was divided into two sections:

1. The “localization” part—using a fat suppressed Gradient echo to obtain (in most cases after several orientation scans) an optimal axial image of the pancreas called “the map” (sequence parameters are listed in table 1, below).

2. The “dynamic collection” part—in which a simple gradient echo sequence was used on the same axial slice that was selected in the “localization” part. The first image was recorded prior to the contrast agent's injection and was taken as the “time zero” (baseline) image. Subsequent images with exactly the same parameters were recorded after the injection in an automated manner (sequence parameters are listed in table 1). The first 40 images were recorded consecutively. Since each scan took 10 seconds to record, the entire collection covered roughly the first 7 minutes after the injection (due to technical limitations the first scan was recorded only 15 minutes after the injection). Three additional scans at time intervals of 70 seconds completed the total time coverage of about 10 minutes post injection (the exact times of the scans appear in table 2 below). Preliminary investigations (covering the first 30 minutes post injection) showed that most of the information is contained in the first 10 minutes, the experiments were limited to this time period. The structure of a typical session is summarized in the following scheme (scheme 1):

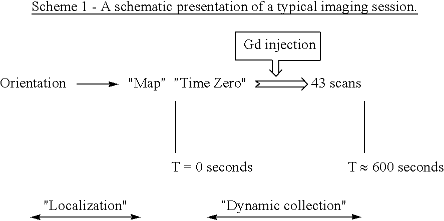

TABLE 1
Sequence parameters of both sections in a typical session. It should be
noted that in order to shorten the time needed for a single scan in the
“dynamic collection”, the number of averages was lowered to two.
In parallel, the slice thickness was doubled to compensate for the
reduction in s/n ratio.

| | | | | Slice | Number |
|---|---|---|---|---|---|
| | | TR | TE | Thickness | Of |
| Section | Sequence name | Msec | msec | mm | Averages |
| Localization | Gefi | 50 | 4.4 | 1 | 8 |
| | Fat_supp | | | | |
| Dynamic | Gefi_bio | 20 | 3.8 | 2 | 2 |
| Collection | | | | | |

TABLE 2
Listing of the exact time for each scan in the “dynamic collection” part
of the MRI session.

| Scan number | Time of scan (seconds) | remarks |
|---|---|---|
| 1 | 0 | “zero time” scan |
| 2 | 15* | Automated collection of scans |
| . | . | |
| . | . | |
| . | . | |
| 41 | 415** | |
| 42 | 495 | |
| 43 | 565 | |
| 44 | 635 | |

*The automated collection was operated immediately after the injection, but took about 10 seconds to start. The exact time of each scan was considered as the time in the middle of the scan. Hence the first scan of the automated collection occurred at 15 seconds.
**Each scan took place 10 seconds after the previous scan.

Data Analysis Procedures

Each experiment yielded a single “map” image and 44 contrast enhancement images. For every pixel in the “dynamic collection” images, a vector of contrast enhanced intensities can be created, which is composed of the intensity value of that pixel over all the scans. Moreover this vector can be correlated to a single pixel in the “map” (since the slices match exactly). Hence, for every pixel identified in the “map”, an intensity profile over time can be created—a graphic presentation of the intensity value vs. the time of the scan for every element in the vector. These intensity curves can be easily converted to enhancement curves, using equation 1.6 (and taking the intensity value of the pixel in the “zero image” as I₀). The same procedure also can be applied to create average enhancement curves for any group of pixels that is identified in the “map” (using the appropriate software to compute the average intensity for those pixels in each “dynamic collection” image).

Average enhancement curves were created for 4 organs:

1. Pancreas.

2. Spleen—which has an important immunological function and shares a common blood supply system with the pancreas.

3. Kidney Cortex—since the kidney in general is sensitive to states of illness.

4. Muscle—taken as an inert marker for which no major changes are anticipated between a healthy and an ill animal.

In practice, ROI's (regions of interest) were drawn around the pancreas, spleen and portions of the kidney cortex and muscle for each animal. Average enhancement curves were then extracted for each organ (i.e. each ROI) according to the above procedure.

Results

Raw Data

For each of 14 animals, an enhancement graph containing the enhancement curves of the 4 organs, was constructed. Soon after their construction, it became clear that all the graphs could be classified into one of two major patterns. Pattern a was taken from a female NOD with blood glucose of 100 mg/dl. Pattern b was taken from a female NOD with blood glucose of 189 mg/dl.

Almost all the animals that belonged to pattern a (with the exception of a single animal) had a blood glucose level below 150 mg/dl (the common threshold for diabetes). At the same, time all the animals that belonged to pattern b had blood glucose level above 150 mg/dl.

The patterns themselves had the following characteristics:

1. Pancreas—the pancreas exhibited an initial rise that eventually reaches a plateau. The enhancement value at the plateau is higher in pattern b compared to pattern a (typical values of 0.6 and 0.3 respectively).

2. Spleen—the spleen exhibits a steep rise followed by a rapid decay. The “height” of the initial rise is higher in pattern a compared to pattern b (typical values of 1.0, 0.6 respectively).

3. Kidney—the kidney demonstrates its regular enhancement profile of an initial rise followed by a decay to a negative enhancement value (a darkening effect due to a shortening of T₂). No clear differences were observed in the kidney between the two patterns.

4. Muscle—the behavior of the muscle was similar to that of the pancreas except that the plateau was reached at longer times. As in the kidney, no significant differences were detected between both patterns.

Analysis of the Enhancement Data

Enhancement curves, although very illuminating, are to some extent, qualitative and descriptive. As explained above, an objective of the system and method of the invention was to correlate the enhancement data to other parameters that are closely connected to the progression of IDDM, namely the blood glucose level and the histological state of the pancreas (the formation of insulitis etc.). In order to do this, the enhancement curves had to be translated to a set of discrete numerical values; in other words, the data needed to be fitted to a parametric function. This procedure was applied to two organs: the pancreas, and the muscle (which was estimated to be an inert organ). Another procedure—cruder and simpler—was applied to the spleen. The choice of the former organs was not only functional, but also practical—the enhancement curves of these organs seemed to obey a simple functional behavior. Both enhancement curves—in their initial phase (“wash in” phase)—showed a rise that reached a steady “plateau”. Hence a dependence of the following type was assumed:

ΛΛE=a(1−e^{-bt}) (4.1)

The “a value” represents the enhancement value at the plateau, or the maximal concentration of the contrast agent in the tissue—a capacity related to histological parameters such as the extracellular volume fraction. At the same time, the “b value” represents the rate in which the enhancement curve reaches the plateau—or the ease by which the contrast agent “leaks” from the blood vessels into the tissue.

It can be seen that the quality of the numerical fitting depends on the scattering in time of the collected points in the enhancement curve—an optimal fitting of “a” requires a lot of points in the plateau, while an optimal fitting of “b” requires a lot of points in the initial rise. Since some of the animals exhibited a steep rise—much faster than the temporal resolution of 10 seconds—it was decided to use the results of the non-linear fitting to equation (4.1) to extract only the “a” parameter. In addition, pancreatic enhancement curves (that showed, in general, a quick rise) were fitted only up to 300 seconds, while the muscle curves were fitted to all of the data up to 635 seconds.

As for the “b” parameter, a different approach was attempted. Instead of a non-linear fitting, which requires very good data, a linear fitting of the first points in time (an “initial rate” fitting) was tried. This approach is justifiable since at very short times equation (4.1) represents a straight line. This can be realized if the equation is expanded in a Mclaurin series to give equation (4.2) as follows:

ΛΛE_{t→0}≈a(1−<1−bt>)=(ab)·t (4.2)

In practice, the first four points of each enhancement curve (of both pancreas and muscle) were fitted to equation (4.2).

Correlating the “a value” to the Blood Glucose Level

Contrast enhancement measurements were taken from 14 animals, of which 4 were normal BALB/c and 10 NOD, with blood glucose levels ranging from 88 to 426 mg/dl. As a first step, the “a values” of the pancreas and muscle were plotted against the blood glucose levels, for both mouse strains. The measurements of 136 mg/dl, 198 mg/dl were performed on the same animal in a time interval of 6 days. The solid line represents the best linear fit of the pancreas data (see below).

The results reveal a clear relation between the “a value” of the pancreas and the blood glucose level in the NOD population. The “a value” increases quite linearly with increasing blood glucose levels.

A linear fitting of the “a value” in the pancreas gave the following phenomenological relation, which shows an R² value of 0.88:

ΛΛa_{pancreas}=0.001·[Glucose]_{blood}+0.234 (4.3)

On the other hand the “a values” in the muscle appeared quite stable. The BALB/C population exhibited similar “a values” to those seen in prediabetic NOD mice, both in the pancreas and in the spleen. These results are in accordance with the hypothesis that the “a values”—representing the space available for the contrast agent in the tissue—will increase as the mice become more diabetic due to processes that accompany the inflammation in the pancreas (formation of edema, increase in the blood vessel permeability, etc.). The muscle, in contrast, can be seen to be unaffected by the inflammatory processes occurring in the pancreas.

These results became even clearer when the mice population was classified into three groups, based on their blood glucose levels. The groups were:

1. BALB/c—4 animals.

2. Pre-diabetic NOD's—5 animals.

3. Diabetic NOD's—5 animals.

The dividing line between groups 2 and 3 was set at 150 mg/dl, which is a common threshold for the NOD model.

The dramatic difference in the mean “a value” between the pre-diabetic and diabetic (an increase of more than 100%) is vivid. In addition, one sees that the mean “a values” of the pre-diabetic are the same as those of the BALB/c—a very plausible outcome since the intact pancreas of the healthy NOD mice should have the same parameters as those of the healthy strain. Yet another feature is the constant value of the mean “a value” in the muscle of all three groups.

The statistical significance of the difference between the three groups was computed by an unpaired Student's t test. The results of this test are summarized below:

TABLE 3
P values of pair comparison for the three groups of pancreatic
P values less than 0.05 indicate that the difference between the
two groups is statistically significant.

| The compared pair | P value |
|---|---|
| Prediabetic NOD − diabetic NOD | 0.007 |
| BALB/c − diabetic NOD | 0.058 |
| BALB/c − prediabetic NOD | 0.717 |

The results demonstrate that the mean pancreatic “a value” of the diabetic group is indeed significantly different than that of the pre-diabetic group. Another plausible result is the high P value of the last pair. These two groups are very similar from the biological point of view—a fact reflected in the high P value obtained for this pair.

Correlating the “a value” to the Pancreatic Histology

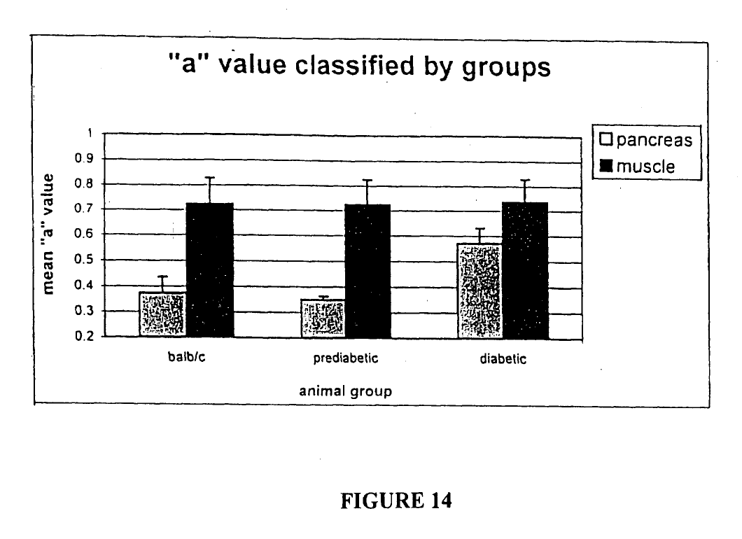

In addition to the blood glucose level, the association of the “a value” with the histological condition of the pancreas in each animal was explored. This was done in view of the basic working hypothesis that changes in the parameters of contrast enhanced images of the pancreas can be attributed to the local inflammatory changes that occur in the pancreas during the progression of IDDM. The histological process is, of course, continuous, but goes through several distinct “stages”. Since it was not possible to quantify the state of the tissue, it was decided to classify all the animals into three categories (according to their pancreatic condition): the intact group, the acute insulitis group and the atrophic group. The classification in practice was based on examining the histological slices taken from the pancreas of each animal (except of 1 that could not be examined due to technical problems). The results of the classification are shown in FIG. 14.

Indeed, the histological composition of each group was associated with the glucose level based classification. In other words, the group of the “intact” pancreas matched exactly the group of the BALB/c, the “acute insulitis” matched the pre-diabetics, and the “atrophic” matched the diabetics. Thus, the mean “a value” of the “acute insulitis” group was similar to that of the BALB/C.

Correlating the “b value” to the Blood Glucose Level

As explained above, the “b value” was extracted from the first four points of the enhancement curve. In practice, a linear fit to these four points was preformed while requiring that the intercept of the linear line would be at the origin (in order to satisfy equation 4.2). As for the “a value”, this procedure was applied to the enhancement curves of both the pancreas and muscle. The quality of these fittings was, in general, rather poor (the average R² value was about 0.6). The derived “b values” were then plotted against the blood glucose level of each animal.

The results show that the “b value”, like the “a value”, tends to increase with increasing blood glucose levels. A linear fit of the “b values” gave rather good results, although inferior than those obtained for the “a values” (the R² value was 0.79). At the same time the muscle, on average, is quite stable. Also, the mean “b values” of the three animal groups (BALB/c, prediabetic and diabetic) were investigated. The results of this approach seem less decisive than those of the “a value” method (for example there were fluctuations of the mean “b value” of the muscle). It was concluded that the “a value” is a more reliable indicator to the stage of IDDM in mice.

Correlating the Splenic Enhancement Curve with the Blood Glucose Level

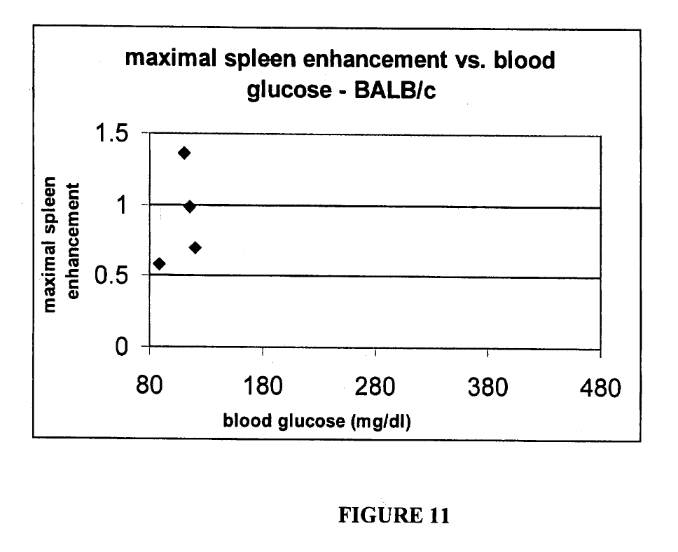

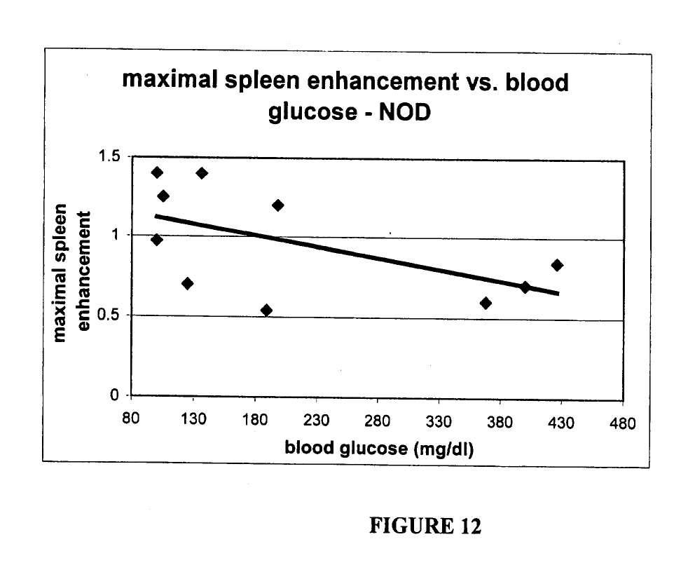

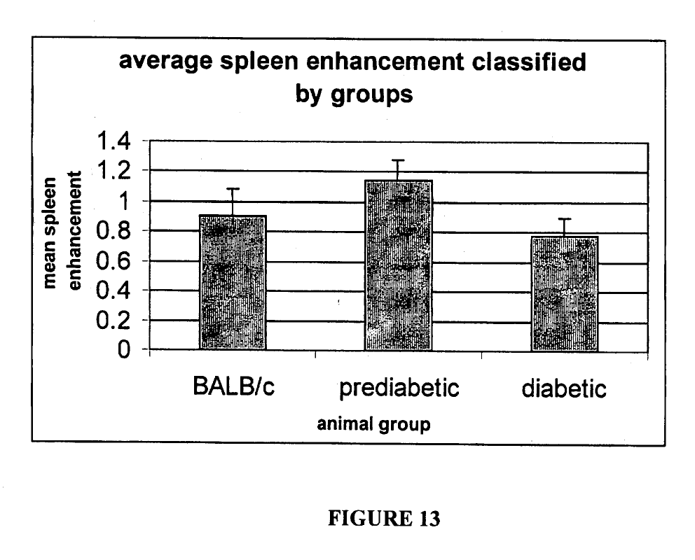

As shown above, the enhancement curve of the spleen was different from those of the pancreas and muscle. Moreover, a simple function to which the enhancement curve of the spleen could be fit, was not found. In order to circumvent this difficulty, a much simpler (but also less accurate) method was used. In this method, the maximal enhancement value of the initial—“wash in”—phase (t<60 seconds) were extracted. This value was plotted against the blood glucose levels according to the same method used for the “a values” and “b values”. The results of these analyses are summarized in FIGS. 11, 12 and 13. In FIG. 12 the solid line represents the linear fit to the splenic data.

The overall results indicate that there is a connection between the enhancement curves obtained for the spleen and the blood glucose level of each animal. More elaborately, the maximal spleen enhancement observed during the “wash-in” phase tends to decrease, as the blood glucose level increases (in contrast to the trend observed in the pancreas). This decrease doesn't seem to follow a linear rule (the R² value of the linear fit was 0.3). Although these findings are less sensitive to the progression of IDDM (compared to the observations made in the pancreas), they suggest that the IDDM process is not limited to the islets, but that the immune tissues may take part systemically.

Conclusions

The contrast enhancement curves of the pancreas and spleen were markedly different for pre-diabetic NOD (and BALB/C) mice on the one hand, and diabetic NOD mice on the other hand. In addition to the visual difference between the enhancement curves, a quantitative way of distinguishing a diabetic from a pre-diabetic pancreas was devised. This was achieved by fitting the experimental enhancement curve of the pancreas to a phenomenological function with two free parameters. One of these parameters was then plotted against the blood glucose level of the same animal (blood glucose was measured independently). A liner dependence of the parametric value (termed the “a value”) on the blood glucose level in the inspected concentration range, was shown. All pre-diabetic NOD mice had “a values” similar to those of the BALB/c mice. Moreover a similar procedure applied to the muscle tissue did not distinguish pre-diabetic from diabetic NOD mice. The conclution is that the histological changes that take place in the pancreas are reflected in the parameters of the contrast-enhanced images, while the intact muscle does not exhibit any significant change. Histological examination of the pancreas revealed that all the NOD mice were “located” on a continuum that range between acute insulitis and complete atrophy of the islets. It is believed that the major MRI changes in the islets take place only with the appearance of insulitis. Consequently, detectable changes in the “a value” of NOD mice that have not yet developed insulitis are not expected.

Imaging of Pancreatic Pathologies in Human Patients

The above results are used to prepare a standard of “a values” and “b values” for human patients. Any suitable standard presentation may be used; graphical or numerical. The procedures described above are applied to a patient for obtaining the patient's “a value” or “b value”. The obtained values are then compared with the standard for receiving information regarding the condition of the patient's pancreas.

It will be appreciated by persons skilled in the art that the present invention is not limited to what has been particularly shown and described hereinabove. Rather the scope of the invention is defined only by the claims which follow:

1. A method for monitoring a pancreatic pathology in a patient comprising the steps of:
obtaining a first magnetic resonance image of an internal body organ using defined sequence parameters;
injecting a contrast agent to the patient;
obtaining a plurality of subsequent contrast enhancement images of the internal body organ using the defined sequence parameters;
creating an intensity curve, by plotting intensity over time, from the plurality of subsequent contrast enhancement images;
converting the intensity curve to an enhancement curve, said enhancement curve having a linear portion and a plateau portion;
extracting an enhancement value at plateau from the enhancement curve; and comparing the enhancement value at plateau to a standard, thereby monitoring the pancreatic pathology in the patient.

2. The method according to claim 1 further comprising the step of obtaining an axial image of the internal body organ prior to the step of obtaining a first magnetic resonance image, said axial image having defined alignment parameters, and wherein the step of obtaining a first magnetic resonance image and the step of obtaining a plurality of subsequent contrast enhancement images are preformed by using the defined alignment parameters.

3. The method according to claim 1 wherein the internal body organ is the pancreas.

4. The method according to claim 1 wherein the internal body organ is the spleen.

5. The method according to claim 1 wherein the step of injecting a contrast agent is preformed by IV injection of the contrast agent to the patient.

6. The method according to claim 1 wherein the contrast agent does not intersect cell membranes.

7. The method according to claim 6 wherein the contrast agent is gadolinium diethylenetriamine pentaacetic acid.

8. The method according to claim 1 wherein the pancreatic pathology is accompanied by changes in vascularity of the pancreas.

9. The method according to claim 1 wherein the pancreatic pathology is IDDM.

10. The method according to claim 2 wherein the step of obtaining an axial image of the internal body organ comprises the steps of:
applying to the internal body organ a fat suppression pulse having a determined pulse offset frequency and a determined bandwidth; and
obtaining a T1 gradient echo image of the internal body organ.

11. The method according to claim 2 wherein the internal body organ is the pancreas.

12. The method according to claim 2 wherein the internal body organ is the spleen.

13. The method according to claim 2 wherein the contrast agent does not intersect cell membranes.

14. The method according to claim 13 wherein the contrast agent is gadolinium diethylenetriamine pentaacetic acid.

15. The method according to claim 2 wherein the pancreatic pathology is accompanied by changes in vascularity of the pancreas.

16. The method according to claim 2 wherein the pancreatic pathology is IDDM.

17. The method according to claim 1 wherein a large portion of the plurality of subsequent contrast enhancement images is obtained at a time correlating to the plateau portion of the enhancement curve.

18. A method for monitoring a pancreatic pathology in a patient comprising the steps of:
obtaining a first magnetic resonance image of an internal body organ using defined sequence parameters;
injecting a contrast agent to the patient;
obtaining a plurality of subsequent contrast enhancement images of the internal body organ using the defined sequence parameters;
creating an intensity curve, by plotting intensity over time, from the plurality of subsequent contrast enhancement images;
converting the intensity curve to an enhancement curve, said enhancement curve having a linear portion and a plateau portion;
extracting an initial rate value of the enhancement curve; and
comparing the initial rate value to a standard, thereby monitoring the pancreatic pathology in the patient.

19. The method according to claim 18 further comprising the step of obtaining an axial image of the internal body organ prior to the step of obtaining a first magnetic resonance image, said axial image having defined alignment parameters, and wherein the step of obtaining a first magnetic resonance image and the step of obtaining a plurality of subsequent contrast enhancement images are preformed by using the defined alignment parameters.

20. The method according to claim 18 wherein the internal body organ is the pancreas.

21. The method according to claim 18 wherein the internal body organ is the spleen.

22. The method according to claim 18 wherein the step of injecting a contrast agent is preformed by IV injection of the contrast agent to the patient.

23. The method according to claim 18 wherein the contrast agent does not intersect cell membranes.

24. The method according to claim 23 wherein the contrast agent is gadolinium diethylenetriamine pentaacetic acid.

25. The method according to claim 18 wherein the pancreatic pathology is accompanied by changes in vascularity of the pancreas.

26. The method according to claim 18 wherein the pancreatic pathology is IDDM.

27. The method according to claim 19 wherein the step of obtaining an axial image of the internal body organ comprises the steps of:
applying to the internal body organ a fat suppression pulse having a determined pulse offset frequency and a determined bandwidth; and
obtaining a T1 gradient echo image of the internal body organ.

28. The method according to claim 19 wherein the internal body organ is the pancreas.

29. The method according to claim 19 wherein the internal body organ is the spleen.

30. The method according to claim 19 wherein the contrast agent does not intersect cell membranes.

31. The method according to claim 30 wherein the contrast agent is gadolinium diethylenetriamine pentaacetic acid.

32. The method according to claim 19 wherein the pancreatic pathology is accompanied by changes in vascularity of the pancreas.

33. The method according to claim 19 wherein the pancreatic pathology is IDDM.

34. The method according to claim 18 wherein a large portion of the plurality of subsequent contrast enhancement images is obtained at a time correlating to the linear portion of the enhancement curve.

35. A method for detecting insulitis in a patient comprising the steps of:
obtaining a first magnetic resonance image of an internal body organ using defined sequence parameters;
injecting a contrast agent to the patient;
obtaining a plurality of subsequent contrast enhancement images of the internal body organ using the defined sequence parameters;
creating an intensity curve, by plotting intensity over time, from the plurality of subsequent contrast enhancement images;
converting the intensity curve to an enhancement curve, said enhancement curve having a linear portion and a plateau portion;
extracting an enhancement value at plateau from the enhancement curve; and
comparing the enhancement value at plateau to a standard, thereby obtaining information regarding the occurrence of insulitis in the patient.

36. The method according to claim 35 further comprising the step of obtaining an axial image of the internal body organ prior to the step of obtaining a first magnetic resonance image, said axial image having defined alignment parameters, and wherein the step of obtaining a first magnetic resonance image and the step of obtaining a plurality of subsequent contrast enhancement images are preformed by using the defined alignment parameters.

37. The method according to claim 35 wherein the internal body organ is the pancreas.

38. The method according to claim 35 wherein the internal body organ is the spleen.

39. The method according to claim 35 wherein the step of injecting a contrast agent is preformed by IV injection of the contrast agent to the patient.

40. The method according to claim 35 wherein the contrast agent does not intersect cell membranes.

41. The method according to claim 40 wherein the contrast agent is gadolinium diethylenetriamine pentaacetic acid.

42. A method for detecting insulitis in a patient comprising the steps of:
obtaining a first magnetic resonance image of an internal body organ using defined sequence parameters;
injecting a contrast agent to the patient;
obtaining a plurality of subsequent contrast enhancement images of the internal body organ using the defined sequence parameters;
creating an intensity curve, by plotting intensity over time, from the plurality of subsequent contrast enhancement images;
converting the intensity curve to an enhancement curve, said enhancement curve having a linear portion and a plateau portion;
extracting an initial rate value of the enhancement curve; and
comparing the initial rate value to a standard, thereby monitoring the pancreatic pathology in the patient.

43. The method according to claim 42 further comprising the step of obtaining an axial image of the internal body organ prior to the step of obtaining a first magnetic resonance image, said axial image having defined alignment parameters, and wherein the step of obtaining a first magnetic resonance image and the step of obtaining a plurality of subsequent contrast enhancement images are preformed by using the defined alignment parameters.

44. The method according to claim 42 wherein the internal body organ is the pancreas.

45. The method according to claim 42 wherein the internal body organ is the spleen.

46. The method according to claim 42 wherein the step of injecting a contrast agent is preformed by IV injection of the contrast agent to the patient.

47. The method according to claim 42 wherein the contrast agent does not intersect cell membranes.

48. The method according to claim 47 wherein the contrast agent is gadolinium diethylenetriamine pentaacetic acid.

49. An MRI system for monitoring a pancreatic pathology in a patient comprising a single volume coil for transmitting and receiving signals from an internal body organ selected from the group consisting of the pancreas, and the spleen.

50. The MRI system according to claim 49 further comprising a spectrometer recording at 4.7 Tesla.
